(19) United States
(12) Patent Application Publication
(10) Pub. No.: US 2025/0041367 A1
(43) Pub. Date: Feb. 6, 2025

(54) BACTERIOPHAGES AND USE THEREOF FOR THE TREATMENT OR PREVENTION OF INFECTION CAUSED BY E. COLI

(71) Applicant: Purdue Research Foundation, West Lafayette, IN (US)

(72) Inventors: Paul EBNER, West Lafayette, IN (US); Zafar HAYAT, Sargodha (PK); Shafiq ur REHMAN, Lahore (PK); Abdul HANNAN, West Lafayette, IN (US)

(21) Appl. No.: 18/793,898

(22) Filed: Aug. 4, 2024

Related U.S. Application Data
(60) Provisional application No. 63/530,660, filed on Aug. 3, 2023.

(51) Int. Cl. A61K 35/76; A61K 9/50; A61P 31/04; C12N 7/00

(52) U.S. Cl. CPC A61K 35/76; A61K 9/5036; A61P 31/04; C12N 7/00; C12N 2795/00021; C12N 2795/00032

(57) ABSTRACT

A composition of bacteriophages comprising at least one phage selected from AHP, AHC, AKA, MIA, MP1, and MP2, wherein the phages are optionally encapsulated; and a method for preventing or treating an infection or a disease caused by avian pathogenic E. coli by administering to a subject a prophylactically or therapeutically effective amount of the composition.

### CROSS REFERENCE TO RELATED APPLICATIONS

This application claims priority to U.S. provisional patent application No. 63/530,660, which was filed Aug. 3, 2023, and which is hereby incorporated by reference in its entirety.

### TECHNICAL FIELD

The present disclosure relates to a bacteriophage-based treatment for infections or diseases caused by E. coli. Particularly, the disclosure relates to compositions of bacteriophages and their use for preventing or treating an infectious disease, such as a disease caused by avian pathogenic E. coli (APEC) in poultry.

### SEQUENCE LISTING

A computer-readable form (CRF) of the Sequence Listing is submitted with this application. The sequence listing is entitled 70146-02_SEQ_LISTING.xml, was generated on Jul. 29, 2024 and is 673,128 bytes in size. The entire content of the sequence listing is incorporated herein by reference in its entirety.

### BACKGROUND

Non-pathogenic E. coli resides in the gastrointestinal tracts of most animals and is generally considered a commensal organism. Some serotypes, however, are pathogenic, and infections with these serotypes typically result in diarrheal disease. Certain types of E. coli are considered highly pathogenic and can cause enterohemorrhagic fever, renal failure, and, in some cases, death. Some E. coli are species-specific, meaning that their pathogenicity is heightened in certain host species. Avian pathogenic E. coli (APEC) is a species-specific E. coli that causes disease in poultry. APEC serotypes, for example, 078, 01, 02, and 05, are commonly associated with APEC infections in poultry.

APEC predominantly colonizes the small intestine of birds, and may cause both systemic and localized infections. Acute APEC infections are characterized by septicemia, while subacute infections may manifest as airsacculitis (air sac infection), perihepatitis (liver infection), or salpingitis (oviduct infection). APEC may also cause egg peritonitis, skin infections, and coligranuloma (Apostolakos et al., 2021, Frontiers in Veterinary Science, 8, 1040). Infections caused by APEC are collectively referred to as avian colibacillosis. Colibacillosis can result in bird mortality rates of 20% to 50% and beyond, depending on the age of the birds at the time of infection, and causes significant economic loss to poultry industries worldwide. Colibacillosis is usually controlled using antibiotics. Unlike many bacterial infections, there are no vaccines available for colibacillosis due to the diversity of APEC O-serotypes.

The use of antibiotics in food animal production for any reason (e.g., for disease treatment, growth promotion, etc.) can lead to the development and spread of antibiotic resistance in bacterial populations, resulting in a reduction in the efficacy of the antibiotic in question. Antibiotics can also accumulate in animal tissues before being excreted. The presence of antibiotic residues in animal tissues may lead to regular, albeit low dose, exposure to antibiotics within human populations, potentially disrupting microbial communities and leading to further antimicrobial resistance. The continued emergence of antibiotic resistance has resulted in the prohibition of the use of some antibiotics in poultry production in several countries and an increase in calls to limit the use of antibiotics. Reducing the use of antibiotics in food animal production has been targeted as one way to effectively limit the use of antibiotics and combat the spread of antibiotic resistance.

Therefore, there is a need to develop an alternative treatment to antibiotics to prevent and control serious avian pathogenic E. coli infections or diseases in animals raised for food, such as poultry. It is an object of the present disclosure to provide such an alternative treatment. This and other objects and advantages, as well as inventive features, will be apparent from the detailed description.

### SUMMARY

Six avian pathogenic E. coli (APEC) phages were isolated from untreated wastewater samples from wastewater treatment plants in central Indiana (USA), livestock/poultry waste samples from the Purdue University Animal Sciences Research and Education Center (West Lafayette, IN, USA). The phages were characterized and named as vB_Eco_AHP (AHP), vB_Eco_AHC (AHC), vB_Eco_AKA (AKA), vB_Eco_MIA (MIA), vB_Eco_MP_1 (MP1), and vB_Eco_MP_2 (MP2).

Provided is a bacteriophage composition for preventing or treating an infection or a disease caused by APEC comprising an effective amount of at least one phage selected from AHP, AHC, AKA, MIA, MP1, and MP2, wherein at least one phage is optionally encapsulated.

In some embodiments, the bacteriophage composition comprises phages AHP, AHC, AKA, MIA, MP1, and MP2.

Provided is a bacteriophage composition for preventing or treating an infection or a disease caused by avian pathogenic E. coli (APEC) comprising an effective amount of at least one isolated phage selected from vB_Eco_AHP (AHP) having a genome comprising a nucleotide sequence of SEQ ID NO: 1, vB_Eco_AHC (AHC) having a genome comprising a nucleotide sequence of SEQ ID NO: 2, vB_Eco_AKA (AKA) having a genome comprising a nucleotide sequence of SEQ ID NO: 3, vB_Eco_MIA (MIA) having a genome comprising a nucleotide sequence of SEQ ID NO: 4, vB_Eco_MP_1 (MP1) having a genome comprising a nucleotide sequence of SEQ ID NO: 5, and vB_Eco_MP_2 (MP2) having a genome comprising a nucleotide sequence of SEQ ID NO: 6, wherein at least one phage is optionally encapsulated.

In some embodiments, the bacteriophage composition comprises phages AHP, AHC, AKA, MIA, MP1, and MP2 comprising the nucleotide sequence of SEQ ID Nos: 1 to 6, respectively.

The phages can be encapsulated using synthetic or natural polymers. In exemplary embodiments, the encapsulated phages are encapsulated with natural polymers. Examples of natural polymers include, but are not limited to, sodium alginate, chitosan, gum, gelatin, collagen, or a combination of two or more thereof. Desirably, the natural polymer can be sodium alginate. In some embodiments, the composition comprises encapsulated and unencapsulated phages in a ratio of about 1:1.

A method for preventing or treating an infection or a disease caused by APFC is also provided. The method comprises administering to a subject a prophylactically or therapeutically effective amount of a bacteriophage composition comprising at least one phage selected from AHP, AHC, AKA, MIA, MP1, and MP2, wherein at least one phage is optionally encapsulated. In exemplary embodiments, the subject is poultry. In exemplary embodiments, the disease caused by APEC infections is colibacillosis.

The prophylactically or therapeutically effective amount of the bacteriophage composition can be administered orally, topically, intratracheally, intramuscularly, or subcutaneously to the subject. The composition can be administered in the form of a suspension, through feed, water, an oral gavage, a spray, or an injection over a period of time ranging from about 1 day to 60 days. The composition can also be administered in ovo. In some embodiments, the prophylactically or therapeutically effective amount is from about 10 PFU to about 10¹³ PFU of each phage per mL of composition, in volumes between 0.1 mL to 5 mL per bird per treatment.

Further provided is a method for preventing or treating an infection caused by APEC comprising administering to a subject a prophylactically or therapeutically effective amount of a bacteriophage composition described herein above.

### BRIEF DESCRIPTION OF THE DRAWINGS

The present disclosure will be more readily understood from the detailed description of embodiments presented below considered in conjunction with the attached drawings of which:

FIG. 1 shows the lytic capacity of phages MP1, AHP, MP2, MIA, AKA, and AHC against the avian pathogenic E. coli (APEC) serotypes for which they are lytic. Asterisks (*) indicate significant difference at P<0.05. Statistical comparisons are within time points.

FIG. 2 shows the lytic capacity of a bacteriophage composition comprising phages MP1 AHP, MP2, MIA, AKA, and AHC and compares the effect of this phage treatment on APEC growth in a liquid medium with the effects of various antibiotics. Asterisks * indicate significant difference at P<0.05. Statistical comparisons are within time points.

FIG. 3 shows the effect of simulated gastric fluid (SGF) on the viability of unencapsulated phages MP1, MP2, AKA, MIA, AHP, and AHC over time. Concentrations of unencapsulated phages exposed to simulated gastric fluid (SGF, grey bars) were significantly lower than those of unencapsulated phages exposed to a 1×PBS control solution (black bars). Asterisks * indicate a significant difference at P<0.05, while “ns” indicates no significant difference. Statistical comparisons are within time points.

FIG. 4 shows the viability of encapsulated phages MP1, MP2, AKA, AHP, MIA, and AHC after exposure to SGF over time. Concentrations of encapsulated phages exposed to SGF (grey bars) for 15, 30, or 60 minutes were not found to significantly differ from those of encapsulated phages exposed to a 1×PBS control solution for the same length of time. Concentrations of encapsulated phages exposed to SGF for 90 minutes were found to be significantly lower than those of encapsulated phages exposed to a 1×PBS control solution for 90 minutes. Asterisks * indicate a significant difference at P<0.05, while “ns” indicates no significant difference. Statistical comparisons are within time points.

FIG. 5 shows the effect of simulated intestinal fluid (SIF) on the viability of unencapsulated phages MP1, MP2, AKA, AHP, MIA, and AHC over time. Except in the case of MP1, concentrations of unencapsulated phages exposed to SIF (grey bars) were not found to differ significantly from concentrations of unencapsulated phages exposed to a 1×PBS control solution at any time point. Asterisks * indicate a significant difference at P<0.05, while “ns” indicates no significant difference. Statistical comparisons are within time points.

FIG. 6 shows the concentrations of phages MP1, MP2, AKA, AHP, MIA, and AHC released from sodium alginate microcapsules after exposure to SIF for 1, 2, or 3 hours (all microcapsules were exposed to SGF for 1 hour before being exposed to SIF). Substantial concentrations of all phages were released from microcapsules after one hour of incubation in SIF. Except in the case of AHP, the concentrations of released phages increased significantly between 1 and 2 hours of incubation with SIF. Across all phage types, no significant difference was observed between the concentration of released phages after three hours of incubation with SIF and the concentration of released phages after two hours of incubation. Asterisks * indicate a significant difference at P<0.05, while “ns” indicates no significant difference.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended by the description of these embodiments. On the contrary, this disclosure is intended to cover alternatives, modifications, and equivalents as may be included within the spirit and scope of this application. As previously noted, while this technology may be illustrated and described in one or more preferred embodiments, the compositions, and methods hereof may vary.

The terms “bacteriophage,” “phage,” “bacteriophages,” and “phages” are used to refer to a functional phage particle or particles, respectively, comprising a nucleic acid genome packaged in a proteinaceous envelope or capsid. The term also refers to portions of the bacteriophage, including, e.g., a head portion, an assembly of phage components or other phage proteins, which provide substantially the same functional activity as the bacteriophage in its entirety.

The term “lytic activity” designates the property of a bacteriophage to cause the lysis of a bacterial cell.

The term “PFU” means “plaque forming unit” and refers to the number of virus particles capable of forming plaques per unit volume, as it is well defined in the art. Lytic bacteriophages lyse the host cell, causing a zone of clearing (or plaque) on a culture plate. Theoretically, each plaque is formed by one phage, and the number of plaques multiplied by the dilution factor is equal to the total number of phages in a given test preparation.

The term “composition of bacteriophages” refers to a composition of bacteriophages, which can be the same or different. A composition comprises at least one of vB_Eco_AHP (AHP), vB_Eco_AHC (AHC), vB_Eco_AKA (AKA), vB_Eco_MIA (MIA), and vB_Eco_MP_1 (MP1) and vB_Eco_MP_2 (MP2). The composition can comprise two, three, four, five, or all six, phages, such as MP1, MP2, AKA, AHP, MIA and AHC, MP1 and MP2, MP1 and AKA, MP1 and AHP, MP1 and MIA, MP1 and AHC, MP2 and AKA, MP2 and AHP, MP2 and MIA, MP2 and AHC, AKA and AHP, AKA and MIA, AKA and AHC, AHP and MIA, AHP and AHC and MIA and AHC. Other examples of combinations include, but are not limited to, MP1, MP2, and AKA; MP2, AKA, and AHP; AKA, AHP, and MIA; or AHP, MIA, and AHC. Yet other examples of combinations include, but are not limited to, MP1, MP2, AKA, and AHO; MP2, AKA, AHP, and MIA; AKA, AHP, MIA and AHC; MP1, AKA, AHP, and MIA; MP1, AHP, MIA, and AHC; MP2, AKA, AHP, MIA, and AHC; Still yet other examples of combinations include, but are not limited to, MP1, MP2, AKA, AHP, and MIA; and MP1, MP2, AKA, AHP, MIA and AHC. All permutations of combinations are contemplated and encompassed. The bacteriophages in a cocktail/composition can be, and preferably are, formulated together, i.e., in a same vessel or packaging, although they can be prepared as parts of kits in which the (or some of the) bacteriophages are formulated or packaged separately and combined when used or administered.

The term “poultry” means any domesticated bird used for food. Varieties include chicken, turkey, geese, ducks, Rock Cornish hens, and game birds such as pheasant, squab, and guinea fowl. Also included are large birds such as ostriches, emus, and rheas (ratites).

The term “treating” is intended to encompass prophylactic treatment as well as a therapeutic treatment.

Bacteriophages are natural predators of bacteria and are highly host specific. Phages can reproduce themselves and kill their bacterial hosts using the host cell's machinery during the lytic phase of their life cycle. Bacteriophages can be simple to isolate and easily propagated. They can have a long shelf life, increasing the potential for affordable scale-up of bacteriophage-based treatment. The phages can be susceptible to various environmental conditions, including temperature and pH. These environmental factors are important in considering bacteriophage-based treatment, as each factor can reduce phage viability and activity. This is true for the production, storage, and application of phages.

Provided is a bacteriophage composition for preventing or treating an infection or a disease caused by avian pathogenic E. coli (APEC). The bacteriophage composition comprises an effective amount of at least one phage selected from vB_Eco_AHP (AHP), vB_Eco_AHC (AHC), vB_Eco_AKA (AKA), vB_Eco_MIA (MIA), vB_Eco_MP_1 (MP1), and vB_Eco_MP_2 (MP2), wherein at least one phage is optionally encapsulated. Some or all of one type of phage can be encapsulated or, when two or more types of phages are combined, some or all of one type of phage can be encapsulated and none of the other type can be encapsulated (i.e., unencapsulated), some or all of every type of phage can be encapsulated, or all of one type can be encapsulated and none of the other type can be encapsulated (i.e., unencapsulated). Any and all permutations of encapsulation/un-encapsulation are contemplated and encompassed.

Provided are encapsulated phages suitable for preventing and treating the infections or diseases caused by APEC in a subject, such as poultry. The phages can be protected by encapsulating them with natural or synthetic polymers. The phages can be encapsulated to protect them from gastric fluid (GF) and therefore are suitable for oral delivery. These polymers protect phages from harsh acidic conditions, which potentially lead to phage inactivation or loss of phage viability. These polymeric encapsulation materials also protect encapsulated phages from low pH levels, digestive enzymes, and bile juices, allowing phages to remain viable during passage through the upper gastrointestinal tract following oral administration. Encapsulation allows the controlled release of phages. After the phages are released, they can enter the lower gastrointestinal tract, where conditions are less detrimental to phage viability, and there adhere to and lyse susceptible host bacteria.

The suitable polymers can be natural polymers. Examples of natural polymers that can be used for encapsulation include, but are not limited to, sodium alginate, chitosan, gums, gelatin, collagen, or a combination of two or more thereof. The natural polymer can be sodium alginate. The phages can be microencapsulated using sodium alginate and CaCl₂).

In certain embodiments, the bacteriophage composition can comprise a combination of two or more phages selected from AHP, AHC, AKA, MIA, MP1, and MP2, wherein phages are optionally encapsulated. In certain embodiments, the bacteriophage composition can comprise an effective amount of phages AHP, AHC, AKA, MIA, MP1, and MP2, wherein the phages are unencapsulated. In certain embodiments, the bacteriophage composition can comprise an effective amount of phages AHP, AHC, AKA, MIA, MP1, and MP2, wherein the phages are encapsulated.

Provided is a bacteriophage composition for preventing or treating an infection or a disease caused by avian pathogenic E. coli (APEC) comprising an effective amount of at least one isolated phage selected from vB_Eco_AHP (AHP) having a genome comprising a nucleotide sequence of SEQ ID NO: 1, vB_Eco_AHC (AHC) having a genome comprising a nucleotide sequence of SEQ ID NO: 2, vB_Eco_AKA (AKA) having a genome comprising a nucleotide sequence of SEQ ID NO: 3, vB_Eco_MIA (MIA) having a genome comprising a nucleotide sequence of SEQ ID NO: 4, vB_Eco_MP_1 (MP1) having a genome comprising a nucleotide sequence of SEQ ID NO: 5, and vB_Eco_MP_2 (MP2) having a genome comprising a nucleotide sequence of SEQ ID NO: 6, wherein at least one phage is optionally encapsulated.

In some embodiments, the bacteriophage composition comprises phages AHP, AHC, AKA, MIA, MP1, and MP2 comprising the nucleotide sequence of SEQ ID Nos: 1 to 6, respectively.

In some embodiments, AHP having a genome comprising a nucleotide sequence of SEQ ID NO: 1. In some embodiments, AHC having a genome comprising a nucleotide sequence of SEQ ID NO: 2. In some embodiments, AKA having a genome comprising a nucleotide sequence of SEQ ID NO: 3. In some embodiments, MIA having a genome comprising a nucleotide sequence of SEQ ID NO: 4. In some embodiments, MP1 having a genome comprising a nucleotide sequence of SEQ ID NO: 5. In some embodiments, MP2 having a genome comprising a nucleotide sequence of SEQ ID NO: 6.

In some embodiments, AHP comprises a genome comprising a sequence as set forth in SEQ ID NO: 1 or having at least 95% identity, more preferably 96%, 97%, 98% or 99% identity to SEQ ID NO: 1. In some embodiments, AHC comprises a genome comprising a sequence as set forth in SEQ ID NO: 2 or having at least 95% identity, more preferably 96%, 97%, 98% or 99% identity to SEQ ID NO: 2. In some embodiments, AKA comprises a genome comprising a sequence as set forth in SEQ ID NO: 3 or having at least 95% identity, more preferably 96%, 97%, 98% or 99% identity to SEQ ID NO: 3. In some embodiments, MIA comprises a genome comprising a sequence as set forth in SEQ ID NO: 4 or having at least 95% identity, more preferably 96%, 97%, 98% or 99% identity to SEQ ID NO: 4. In some embodiments, MP1 comprises a genome comprising a sequence as set forth in SEQ ID NO: 5 or having at least 95% identity, more preferably 96%, 97%, 98% or 99% identity to SEQ ID NO: 5. In some embodiments, MP2 comprises a genome comprising a sequence as set forth in SEQ ID NO: 6 or having at least 95% identity, more preferably 96%, 97%, 98% or 99% identity to SEQ ID NO: 6.

The composition comprises the encapsulated and unencapsulated phages in various ratios. In certain embodiments, the encapsulated and unencapsulated phages are in a ratio of about 1:1, in which case the encapsulated phage can be of a single type of phage or a combination of phages, in which case less than all of a given type of phage can be encapsulated or all of a given type of phage can be encapsulated. When a combination of phages is encapsulated, the degree to which one type of phage is encapsulated can be the same as, or different from, the degree of encapsulation of one or more other types of phage(s) in the combination.

The bacteriophage composition comprising a combination of encapsulated and unencapsulated phages can acts in such a way that the unencapsulated phages can be immediately active after administration i.e. active in pre-stomach portions of the digestive tract, and the encapsulated phages can be protected until they pass through the stomach. After which point the phages can be released from encapsulation, and act at the intestinal sites of infection (i.e., act in the hindgut).

A method for preventing or treating an infection or a disease caused by APEC is further provided. The method can comprise administering to a subject a prophylactically or therapeutically effective amount of a bacteriophage composition comprising at least one phage selected from AHP, AHC, AKA, MIA, MP1, and MP2, wherein at least one phage is optionally encapsulated.

In certain embodiments, the composition can comprises the phages AHP, AHC, AKA, MIA, MP1, and MP2, wherein the phages are optionally encapsulated. The bacteriophage composition can comprises a combination of encapsulated phages and unencapsulated phages in a ratio of about 1:1. In certain embodiments, the composition can comprise the phages AHP having a genome comprising a nucleotide sequence of SEQ ID NO: 1, AHC having a genome comprising a nucleotide sequence of SEQ ID NO: 2, AKA having a genome comprising a nucleotide sequence of SEQ ID NO: 3, MIA having a genome comprising a nucleotide sequence of SEQ ID NO: 4, MP1 having a genome comprising a nucleotide sequence of SEQ ID NO: 5, and MP2 having a genome comprising a nucleotide sequence of SEQ ID NO: 6, wherein the phages are optionally encapsulated.

Another method for preventing or treating an infection or a disease caused by APEC is provided. The method can comprise administering to a subject a prophylactically or therapeutically effective amount of a bacteriophage composition comprising phages AHP, AHC, AKA, MIA, MP1, and MP2, wherein at least one phage is optionally encapsulated. The bacteriophage composition can comprise a combination of encapsulated phages and unencapsulated phages in a ratio of about 1:1.

Further provided is a method of preventing or treating an infection or a disease caused by APEC. The method comprises administering to a subject a prophylactically or therapeutically effective amount of (i) a bacteriophage composition described herein and (ii) one or more other prophylactic or therapeutic agents. The other prophylactic or therapeutic agent can be any suitable prophylactic or therapeutic agent. In exemplary embodiments, the other prophylactic or therapeutic agent can be a suitable antibiotic agent, such as an antibiotic agent selected from the antibiotic agents known to prevent or treat an infection caused by APEC.

Examples of antibiotic agents include, but are not limited to, ampicillin, doxycycline, neomycin, tilmicosin, amoxicillin, enrofloxacin, ciprofloxacin, norfloxacin, streptomycin, gentamycin, kanamycin, neomycin, cefotoxamin, tetracycline, and sulfomethoxazole.

The other prophylactic or therapeutic agent(s) can be administered simultaneously or sequentially, by the same or a different route, to achieve the desired effect. When administered sequentially, the bacteriophage composition can be administered first, and the other prophylactic or therapeutic agent(s) can be administered second, or vice versa. The bacteriophage composition described herein and one or more other prophylactic or therapeutic agents can be administered from a single composition or as two or more separate compositions, such as by the same or different routes. The prophylactic or therapeutic agent is administered in an amount to provide its desired prophylactic or therapeutic effect. The effective dosage range for each prophylactic or therapeutic agent is well-known in the art or can be determined in accordance with dosage range-determining methods known to those of ordinary skill in the art, and the prophylactic or therapeutic agent can be administered to a subject in need thereof within such established ranges. The bacteriophage composition described herein, and one or more other prophylactic or therapeutic agents can be administered together as a single unit dose or separately as multi-unit doses, wherein the bacteriophage composition can be administered before the other prophylactic or therapeutic agent(s) or vice versa. One or more doses of the composition and/or one or more doses of the prophylactic or therapeutic agent can be administered.

The treatments using the bacteriophage composition comprising the combination of phages can increase the spectrum of the treatment by providing more coverage across different APEC serotypes and thus can have a higher potential to control bacterial growth. Additionally, it can reduce the impacts of phage resistance as there would be a lower probability of a bacterial strain developing resistance simultaneously against multiple distinct phages in the treatment.

A “subject” can be any animal susceptible to infection with APEC, such as poultry. Example of poultry includes, but are not limited to, domesticated birds such as chickens, ducks, turkeys, geese, or Rock Cornish hens; game birds such as pheasant, squabs, guinea fowl, peafowl, grouse, or quail; and large birds such as ostriches, emus or rheas. In exemplary embodiments, the subject can be a chicken.

An infection caused by APEC result in colibacillosis. Colibacillosis includes extra-intestinal bacterial infections, salpingitis (infection of fallopian tubes), pericarditis, perihepatitis (liver infection), airsacculitis (air sac infection), egg peritonitis, septicemia, skin infections, and coligranuloma.

In certain embodiments, the bacteriophage composition is formulated in which the bacteriophage is present in an amount to provide a multiplicity of infection (MOI; as determined by the concentration of plaque forming units (PFU) relative to the concentration of bacteria) of about 0.01 to about 1000 upon administration of the composition to a subject in need thereof. In certain embodiments, the concentration of one or more bacteriophage(s) can be combined to form a composition comprising at least about 10 PFU to about 10¹³ PFU per mL of composition, such as from about 10 PFU to 10¹³ PFU or 10 PFU to about 10¹³ PFU of each phage per mL of composition. In certain embodiments, the total concentration of all bacteriophages within the composition can be between about 10 and about 10¹³ PFU per mL of preparation. In certain embodiments, the concentration of the individual bacteriophage within the composition can be between about 10 and about 10¹³ PFU per mL of preparation.

As noted above, the therapeutically or prophylactically effective amount of the bacteriophage composition can be administered to the subject. The bacteriophage composition comprises unencapsulated phages and/or encapsulated phages, or a combination thereof. For example, and without limitation, the therapeutically or prophylactically effective amount can be administered to a subject in need of treating or preventing the infection or disease caused by APEC. An effective amount of the bacteriophage composition can be administered to the subject prophylactically (e.g., before disease onset) or therapeutically (e.g., concurrently or after disease onset). In certain embodiments, the effective amount can be administered orally to the subject prior to, during, or following APEC exposure, which may lead to infection, in a single or multiple administrations. The effective amount can, for example, include about 10 PFU to about 10¹³ PFU of each phage per ml of composition, in volumes between 0.1 mL to 5 mL per bird per treatment. The total effective amount of the bacteriophage composition can be administered in a single dose or over multiple doses and can, at the practitioner's discretion.

In certain embodiments, a bacteriophage composition can be administered to a subject at about 10 PFU to about 10¹³ PFU of each phage per ml of composition, in volumes between 0.1 mL to 5 mL per bird per treatment. The bacteriophage composition can be administered at least once, twice, thrice or more times daily. The bacteriophage composition can be administered over a period from about 1 day to about 60 days.

The subject can have an infection or disease, such as colibacillosis, caused by APEC. The effective amount of the bacteriophage composition can be administered to the subject orally. As used herein, the term “administering” and its formatives generally refer to any and all means of introducing a composition to the subject, including, but not limited to, by oral, topical, intravenous, subcutaneous, intratracheal, intramuscular, and like routes of administration. The composition can also be administered in vivo. In exemplary embodiments, the bacteriophage composition can be administered by oral gavage, such as by using a Monoject Curved Tip Syringe. By way of other examples, the bacteriophage composition can be administered as a capsule or in the form of feed or water.

In certain embodiments, a bacteriophage composition comprises a prophylactically or therapeutically effective amounts of one or more bacteriophages selected from AHP, AHC, AKA, MIA, MP1, MP2, and any combination of two or more of the foregoing and a pharmaceutically acceptable carrier, excipient, or diluent.

Effective amounts can range, for example, from about 10 PFU to 10¹³ PFU of each phage per ml of composition, in volumes between 0.1 mL to 5 mL per bird per treatment. The total effective amount of the bacteriophage composition can be administered in single or divided doses and can, at the practitioner's discretion, fall outside of the typical ranges given herein.

Six bacteriophages were isolated to develop a bacteriophage-based treatment for infection or disease caused by APEC, for example, colibacillosis, in a subject. A suitable subject can be a chicken. The phages were isolated from untreated wastewater samples obtained from a wastewater treatment plants in central Indiana (USA), livestock/poultry waste samples from the Purdue University Animal Sciences Research and Education Center (West Lafayette, IN, USA), area using the methods as described in Wall et al., 2010, Applied and Environmental Microbiology, 76 (1), 48-53, which is hereby specifically incorporated by reference for its teachings regarding same. The individual ability of isolated bacteriophages to lyse ten different APEC serotypes was assessed by conducting spot assays using all ten APEC serotypes. This host-spectrum analysis indicated that the bacteriophages (i.e., MP1, MP2, AHP, AKA, AHC, and MIA) that were isolated, purified, and assessed had a broad host range and some degree of lytic capacity against at least two different APEC serotypes, with four of the phages lysing four or more distinct APEC serotypes. The degree of lysis was measured using a four-point scale. A score of 0 indicated no lysis, 1.0 indicated some lysis, 2.0 indicated moderate lysis, and 3 indicated complete lysis. Considered together, the six phages had lytic activity against 90% (9/10) of the targeted O-serotype APEC strains. All phages lysed APEC serotype O2:H4 to some degree.

The further lytic activity analyses of six bacteriophages indicated that these phages were highly lytic against their respective APEC hosts. Specifically, when compared to APEC cultures incubated without phages, MP2, and AHP-treated APEC cultures were found to have significantly lower (P<0.05) concentrations of their respective APEC hosts at 1, 2, 3, and 4 hours post-treatment. Significantly lower (P<0.05) concentrations of APEC were observed in host APEC cultures co-incubated with either AKA, AHC, or MIA than in untreated APEC cultures at 2, 3, and 4 hours post-treatment (Error! Reference source not found.). As each bacteriophage was tested with its respective APEC serotype, differences in the length of time it took for each of the phages to cause a significant reduction in the concentration of their APEC host may have been the result of differences in the growth rates of the APEC serotypes or differences in phage adsorption and other growth kinetics. Individual bacteriophage types were co-incubated with their respective APEC hosts at an MOI of 10. The lytic capacity of a combination of AHP, AKA, AHC, MIA, MP1, and MP2 was also assessed. The results of this experiment indicated that APEC cultures treated with these six phages in combination had significantly lower (P<0.05) APEC concentrations at 2, 3, and 4 hours post-treatment in comparison to untreated APEC cultures. The phage combination was co-incubated with APEC at an MOI of 10.

The genome of each phage was sequenced, and AHP, AKA, AHC, MIA, MP1, and MP2 were further assessed to determine the viability of each phage in different environments. During these assessments, phages were exposed to simulated gastric fluids (SGF; pH 2.5) and to simulated intestinal fluids (SIF; pH 7).

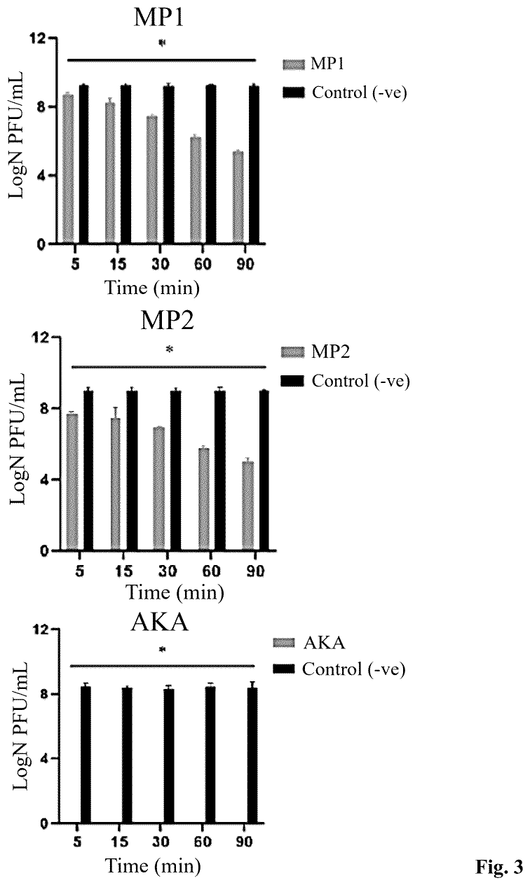

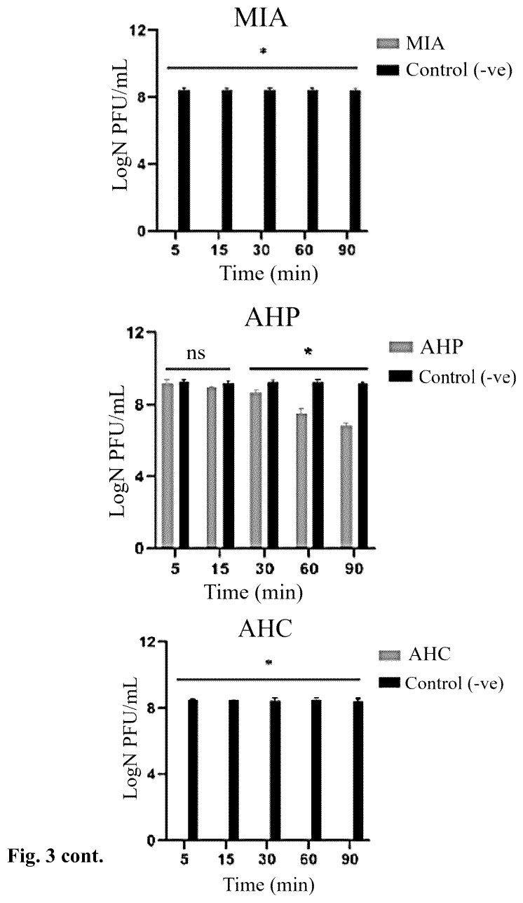

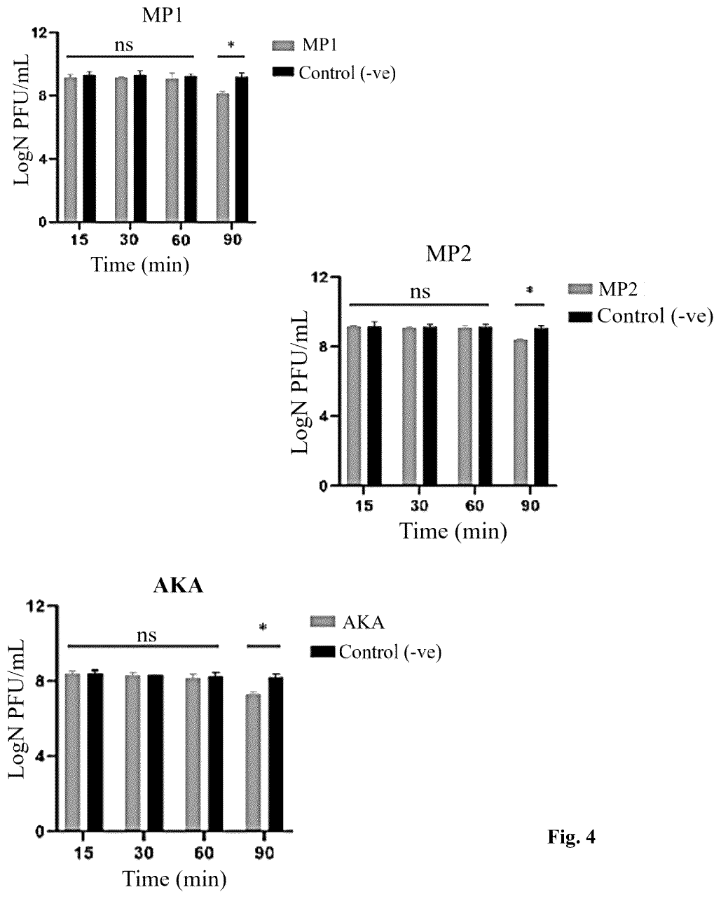

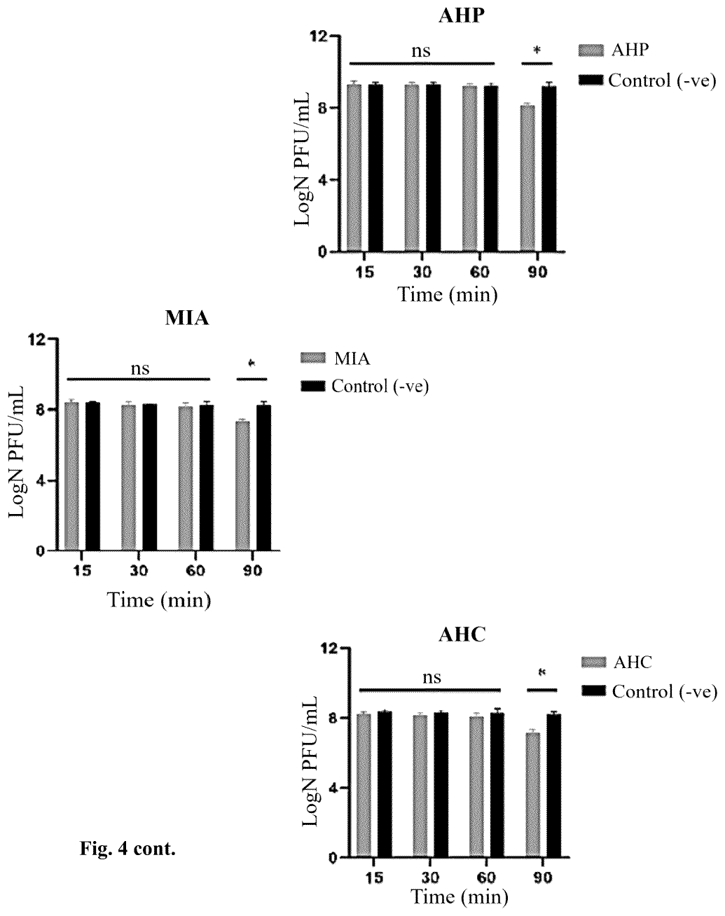

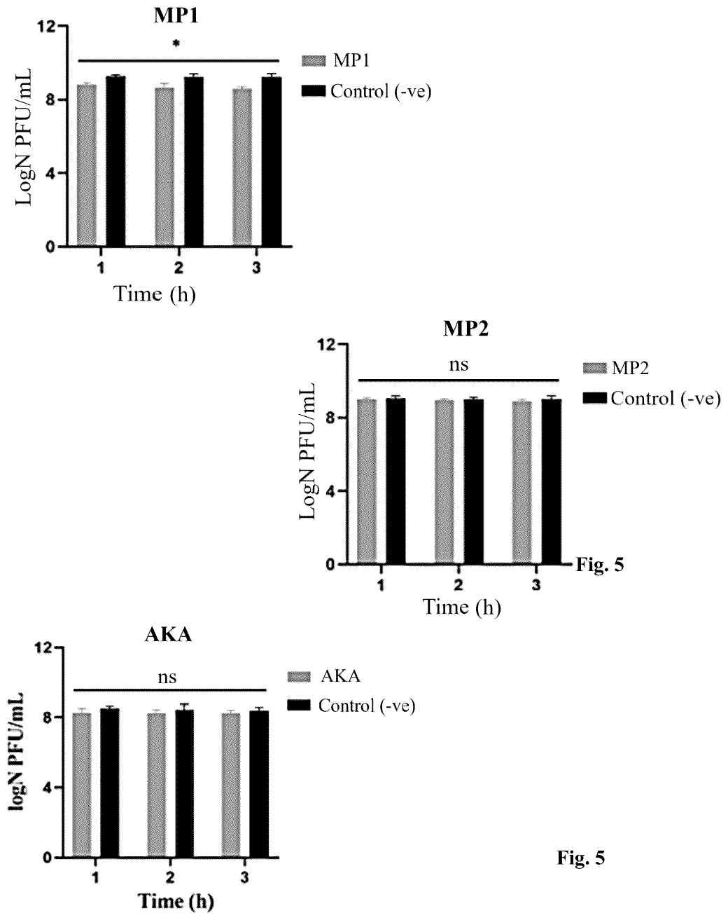

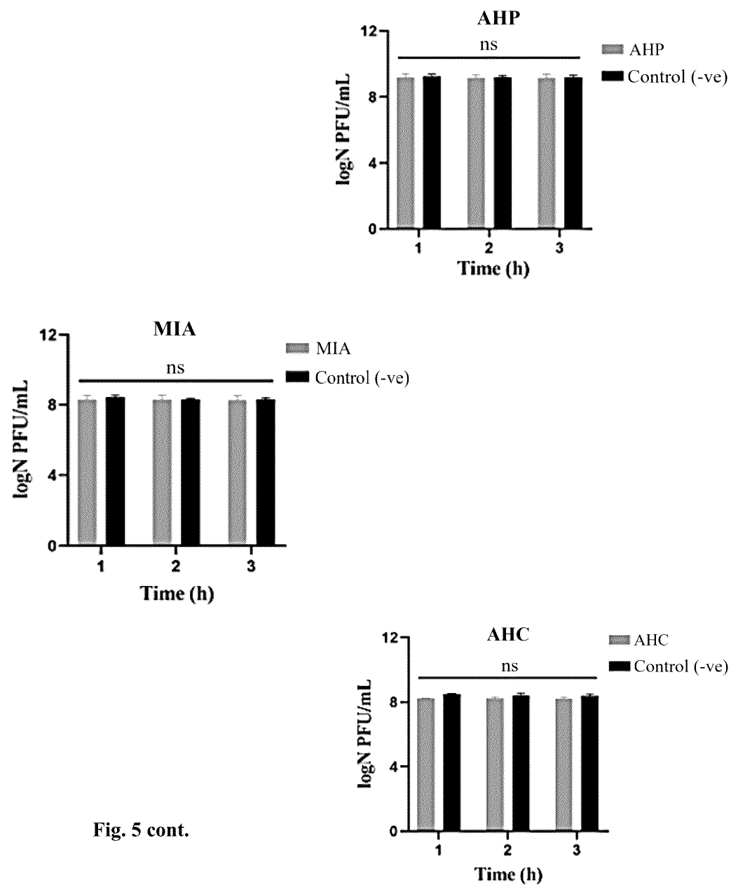

APEC colonizes the hindgut in poultry, and a successful intervention against APEC must be able to effectively reduce concentrations of APEC within the hindgut. Phage viability was tested by incubating unencapsulated and encapsulated phages in both SGF and SIF. Except in the case of MP1, no significant differences (P>0.05) were observed between the viability of unencapsulated phages exposed to SIF and the viability of unencapsulated phages exposed to a PBS control solution after 1, 2, and 3 hours (FIG. 5). However, phage concentrations were typically significantly lower (P<0.05) in samples of unencapsulated phages exposed to SGF for 5, 15, 30, 60, and 90 minutes than in samples of unencapsulated phages exposed to a PBS control solution for the same time intervals (FIG. 3). Unencapsulated, AKA, MIA, and AHC were not detectable after exposure to SGF for 5 minutes. In contrast, the viability of encapsulated phages exposed to SGF for 15, 30, and, in some cases, 60 minutes was not found to significantly differ from the viability of encapsulated phages exposed to a PBS control solution for the same lengths of time (FIG. 4). The viability of encapsulated phages exposed to SGF for 90 minutes was significantly lower (P<0.05) than that of encapsulated phages exposed to a PBS control solution for 90 minutes, but concentrations of the SGF-exposed encapsulated phages remained above 10⁶ PFU/mL. These results suggest that the viability of unencapsulated phages is typically significantly affected by the intestinal environment and that encapsulated phages were better able to withstand the gastric environment than unencapsulated phages.

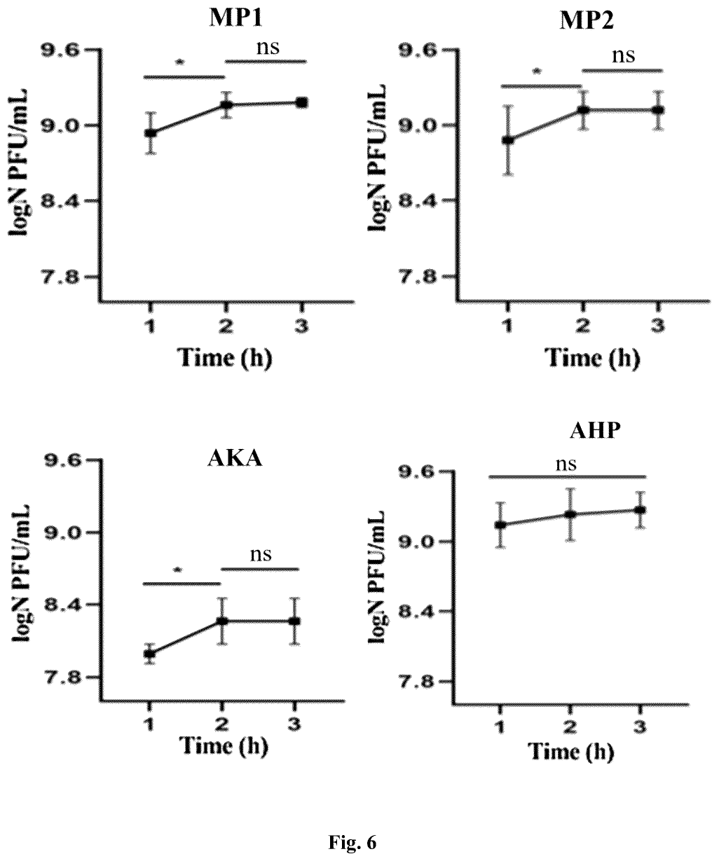

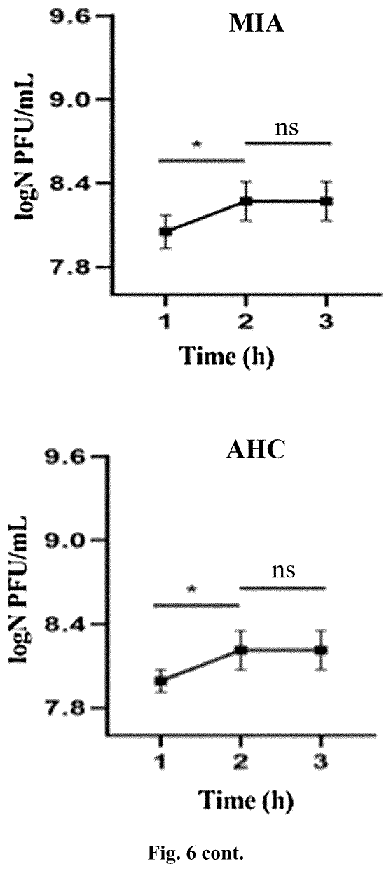

The phages AHP, AKA, AHC, MIA, MP1, and MP2 were encapsulated and exposed to SIF for varying lengths of time in order to measure the concentrations of these phages released into the environment under these conditions. Results indicated that, after exposure to SIF for 1 hour, encapsulated phages had been released into the environment and were present at concentrations above 7.7 log₁₀ PFU/mL (FIG. 6). In most cases, the concentrations of phages in the environment (i.e., phages released from microcapsules) after 2 hours of SIF exposure were significantly higher (P<0.05) than those observed after 1 hour of exposure to SIF. No significant differences were observed between the concentrations of phages in the environment after 2 hours of SIF exposure versus after 3 hours of SIF exposure (FIG. 6).

The efficacy of phages was tested in vivo in chickens experimentally challenged with APEC. The chickens were divided into four groups: a treatment group in which chickens were neither challenged with APEC nor administered phages, a treatment group in which chickens were challenged with APEC but did not receive phages, a treatment group in which chickens were not challenged with APEC but were administered phages, and a treatment group in which chickens were both challenged with APEC and treated with phages. All phage-treated chickens received a composition described herein. The combination of encapsulated and unencapsulated phages showed greater reductions in bacterial colonization as the unprotected phages can immediately attach to and infect APEC in the lungs and proximal sections of the GIT when encapsulated phages can survive the gastric environment and reach distal areas of the GIT. When used alone, on the other hand, unencapsulated phages may not survive into the lower portions of the gastrointestinal tract, and encapsulated phages may not be able to effectively attach to host bacteria in the upper gastrointestinal tract, thereby limiting the impact of these phages on host bacterial concentrations.

Concentrations of APEC in the cecal contents and lung tissues of chickens in all treatment groups were measured. At 1 day post-challenge, concentrations of APEC in the cecal contents of challenged, phage-treated chickens were not found to be significantly different than those of challenged, untreated chickens. However, significantly lower (P<0.05) concentrations of APEC were observed in lung samples from challenged, phage-treated versus challenged, untreated chickens at 1 day post-challenge. APEC concentrations in both cecal content and lung samples were significantly lower (P<0.05) in challenged, phage-treated versus challenged, untreated chickens at 2 days post-challenge. At 4 days post-challenge, concentrations of APEC in both the cecal contents and lungs of challenged, phage-treated birds were again significantly lower (P<0.05) than those in the cecal contents and lungs of challenged, untreated birds. The concentrations of APEC were frequently below detectable limits in lung samples from challenged, phage-treated chickens, and, at 4 days post-challenge, APEC was not detected in any of the lung samples taken from challenged, phage-treated chickens. Additionally, APEC was not detected in the lungs of either unchallenged, untreated or unchallenged phage-treated chickens at any sampling point. Cecal samples from unchallenged, phage-treated chickens were also not found to have detectable levels of APEC at any time point. A limited number of cecal samples from unchallenged, untreated chickens had detectable concentrations of APEC at 4 days post-challenge, though no APEC was detected in samples from this treatment group at 1 or 2 days post-challenge. Lung tissues and the surrounding environment are thought to have little impact on phage viability. Unencapsulated phages can act more immediately in the lungs, causing rapid bacterial lysis and more immediate reductions in bacterial concentrations. Additionally, in most cases, APEC was not recovered from the lungs of phage-treated birds, whereas concentrations of APEC in the lungs of untreated birds was 4.81 log₁₀ CFU/g. The phage treatment effectively controlled APEC colonization and replication in the ceca and lungs of APEC-challenged chickens.

Thus, the phage compositions disclosed herein can successfully control colibacillosis in chickens and have several advantages over traditional antibiotics, including, but not limited to, the control of infections or diseases caused by antibiotic-resistant bacteria.

### EXPERIMENTAL

### Bacterial Serotypes

Five different O-antigen-based serotypes of Escherichia coli (E. coli), namely O78, O2, O1, O5, and O6, account for 85% of avian colibacillosis infections associated with APEC. Three strains of O78, two strains of O2, two strains of O1, one strain of O5, and one strain of O6 were used to assess the lytic capacity of wild-type phages described below. These ten strains were then stored in 20% (v/v) glycerol at −80° C. until use.

### Bacteriophage Isolation and Purification

Untreated wastewater from wastewater treatment plants in central Indiana, livestock/poultry waste from the Purdue University Animal Sciences Research and Education Center (West Lafayette, IN USA), and duck excreta, chicken cecal contents, and chicken excreta from commercial processing facilities were used as samples to isolate APEC phages as described in Wall et al., 2010, Applied and Environmental Microbiology, 76 (1), 48-53, which is hereby specifically incorporated by reference for its teachings regarding same.

Solid waste samples (1 g) were suspended in 10 mL 1×PBS (137 mM NaCl, 2.7 mM KCl, 8 mM Na₂HPO₄, 2 mM KH₂PO₄; pH=7) and homogenized by vortexing. This suspension was centrifuged (2,650×g) for 20 minutes to sediment solids. The supernatant was then filtered through 0.45 μm filters. The filtrate was mixed in equal volumes (1:1) with log-phase cultures of individual APEC strains in 2× LB broth and incubated at 37° C. overnight with shaking. These overnight cultures were centrifuged (2,650×g) for 20 minutes, and the supernatants were filtered through 0.45 μm filters. Phages present in the filtrates of the overnight cultures were detected by standard spot assay. To perform spot assays, a mixture of 67.5 μL of CaCl₂) (1M), 100 μL of an individual APEC strain at log-phase growth, and 4 mL of LB overlay was pour-plated onto an LB agar plate. Once the overlay solidified, 5 μL droplets of each filtrate were applied to the top layer of the plate, allowed to dry, and incubated at 37° C. overnight. The resulting plaques were lifted and transferred to 1 mL of 1×PBS. This phage/PBS solution was serially diluted and then used to perform a plaque assay. Plaques were purified twice more by plaque assay. Phage stocks were then generated by combining the thrice-purified plaques with 100 mL of log-phase growth APEC, incubating overnight at 37° C. with shaking, centrifuging this overnight culture at 2650×g, and filtering the supernatant through 0.45 μm filters. The filtered supernatant was treated with chloroform (10% v/v) to kill any remaining bacteria and stored at −80° C. until further use.

### Phage Characterization:

### A] Lytic Capacity of Individual Phages

Individual APEC strains (100 μL) were inoculated into 15 mL LB media in separate tubes (two tubes per strain) and incubated at 37° C. in a shaking incubator. Once the bacterial cultures reached an OD₆₀₀ 0.18-0.22, the individual bacteriophage types (MOI=10) were added to one tube from the pair of tubes inoculated with their respective host APEC strain. The other tubes of bacterial culture were left untreated and served as negative controls. Bacterial growth was assessed by measuring absorbance (OD₆₀₀) in all tubes every hour for 4 hours. The experiment was repeated three times.

### B] Lytic Capacity of Phage

The six APEC strains were individually inoculated into LB broth and incubated at 37° C. until they reached log-phase growth. Phages were added to the log-phase culture of their APEC host at an MOI of 10, and subsequent bacterial concentrations were determined by measuring the absorbance (OD₆₀₀) of the culture at 1, 2, 3, and 4 hours post-phage application. One flask containing only bacterial culture was included as a negative control. Additional flasks containing both bacterial cultures and antibiotics (specifically, gentamycin sulfate, kanamycin sulfate, nalidixic acid, or chloramphenicol) at their respective minimum inhibitory concentrations (National Antibiotic Resistance Monitoring System (NARMS) 2019, antibiotics tested by NARMS) were included as positive controls. These lytic capacity experiments were conducted in triplicate for each of the phage types and their respective host APEC strains.

### C] Host Range Characterization

The host range of each bacteriophage type was determined by performing spot assays using each of the ten distinct APEC serotypes mentioned above. Spot assays were performed as known in the art (Barrow et al., 1998, Clinical and Diagnostic Laboratory Immunology, 5 (3), 294-298). 100 μL aliquots of log-phase bacterial cultures were combined with 67.5 μL 1M CaCl₂) and 4 mL LB overlay, mixed briefly, and poured over LB agar plates. Once the overlay had solidified, 5 μL aliquots of individual phage types were applied (i.e., spotted) onto the plates, and plates were incubated at 37° C. overnight. Plates were assessed for clearings the following day and the degree of lysis was measured using a four-point scale in which 0 indicated no lysis, 1.0 indicated some lysis, 2.0 indicated moderate lysis, and 3 indicated complete lysis. Spot assays were conducted in triplicate for each phage and APEC strain.

### D] Whole Genome Sequencing

Phage DNA was extracted using a phenol-chloroform-isoamyl (PCI) method, as well-known in the art (Sambrook and Russell, 2006, CSH Protocols, 2006 (1), 4455) with some modifications (Alvi et al., 2020, Archives of Virology, 165 (6), 1289-1297). Individual phage types (1 mL) were combined with RNase A (100 ug), DNase I (1U), and 1M MgCl₂ (12.5 μL) and incubated at 37° C. for 3 hours. Nucleases were further denatured by incubating the mixture at 75° C. for 20 minutes in a water bath. The solution was then combined with 2.5 μL (20 mg/mL) proteinase K, 50 μL of 10% SDS, and 40 μL of 0.5M EDTA and incubated at 55° C. in a water bath for 1 hour with gentle mixing by inverting tubes every 20 minutes. Each of the lysate and reagent mixtures (1107 μL total per mixture) was divided equally into two microfuge tubes (553.5 μL per tube) and an equal amount (1:1) of PCI (553.5 μL; Invitrogen; Waltham, MA, USA) was added to each tube. The samples were gently mixed by inverting the tubes and were then centrifuged at 13,000×g for 10 minutes. The top layer was carefully transferred to a new microfuge tube. Sodium acetate (3M; 50 μL) and 1 mL of ice-cold 95% (v/v) ethanol were added to these tubes and samples were incubated on ice for 10 minutes and then centrifuged at 13,000×g for 10 minutes. The resulting pellet was washed with 500 μL of 70% (v/v) ethanol by pipetting and centrifuged at 13,000×g for 5 minutes. The supernatant was discarded, and the washing step was repeated. After discarding the supernatant the second time, the samples were air-dried by inversion onto blotting paper. The dried pellet was dissolved in 40 μL of DNase/Rnase-free ultrapure distilled water (Invitrogen), and whole-genome sequencing was performed on an Illumina MiSeq next-generation sequencing system.

### E] Encapsulation of Phages

Bacteriophages were microencapsulated within sodium alginate microcapsules as well-known in the art with some modifications (Krasaekoopt et al., 2004, International Dairy Journal, 14 (8), 737-743). In short, sodium alginate (4 g) was mixed with 400 mL of a bacteriophage filtrate to make a 1% (w/v) suspension. This suspension was allowed to gel at room temperature for 4 hours and was then processed through a Buchi Encapsulator-B395 set at 200 Hz (frequency) and 1000V (electrode) with an aperture of 0.3 mm. The resulting microcapsules were collected in a beaker containing a hardening solution (100 mM CaCl₂)) with continuous stirring to avoid clumping of microcapsules. Microcapsules were allowed to sit in the 100 mM CaCl₂) solution to harden for 30 minutes and were then washed three times with SM buffer. Washed microcapsules were stored in SM buffer in 50 ml conical tubes at 4° C. until further use. Each of the six phage types was individually microencapsulated in this manner.

### F] Encapsulation Efficiency

To assess the efficiency of microencapsulation, the concentrations of microencapsulated phages were compared to pre-encapsulation concentrations as well-known in the art (Yongsheng et al., 2008, Applied and Environmental Microbiology, 74 (15), 4799). To determine bacteriophage concentrations in microcapsules, microcapsules (0.5 g) were dried by contact with filter paper and then dissolved in 4.5 mL of dissolving solution (50 mM sodium citrate, 0.2M sodium bicarbonate, and 50 mM Tris-HCl; pH 7.5). Bacteriophage concentrations were then quantified by standard plaque assay as described previously. Encapsulation efficiency (EE %) was calculated as:

EE %=encapsulated phage concentration/pre-encapsulation phage concentration

### Analysis of Phage Stability and Release in Simulated Body Fluids:

### A] Phage Stability in Simulated Gastric Fluid (SGF)

The stability of bacteriophages in gastric environments was tested by exposing both encapsulated and unencapsulated bacteriophages to simulated gastric fluid (SGF; 0.32% pepsin (w/v) in 0.2% NaCl; pH 2.5) and to 1×PBS (Moghtader et al., 2016). In this experiment, unencapsulated phages (0.5 mL, in concentrations ranging from 1×10⁹ to 4×10⁸ PFU/mL depending on the phage type) were added to 4.5 mL of either SGF or PBS (5 replicates per treatment) and incubated at 41° C. This temperature was chosen as it is a typical internal temperature for chickens. Encapsulated phages (0.5 g, in concentrations ranging from 2×10⁹ to 2×10⁸ PFU/mL depending on the phage type) were also added to 4.5 mL of either SGF or PBS (5 replicates per treatment) and incubated at 41° C. Aliquots (100 μL) were collected from each unencapsulated phage sample after 5, 15, 30, 60, and 90 minutes of exposure to SGF or PBS. These aliquots were serially diluted, and phage concentrations were measured by standard plaque assay. At 15, 30, 60, and 90 minutes post-SGF or PBS exposure, microencapsulated phages were washed with SM buffer to remove residual SGF or PBS and then dissolved in a dissolving solution. Aliquots of the dissolved microcapsules were serially diluted and phage concentrations were measured by standard plaque assay. This experiment was conducted in triplicate for each phage type. Samples containing SGF and unencapsulated bacteriophages were serially diluted without washing and phage concentrations were measured by standard plaque assay as described above. Concentrations of viable phages after exposure to SGF or PBS for a given length of time were then compared.

### B] Phage Stability in Simulated Intestinal Fluid (SIF)

To assess phage stability in intestinal fluids, 0.5 mL of unencapsulated phages were added to 4.5 mL of simulated intestinal fluid (SIF; 0.1% (w/v) bile salt and 0.4% (w/v) pancreatin in 50 mM KH₂PO₄; pH 7) and incubated at 41° C. As a negative control, 0.5 mL of unencapsulated phages were added to 4.5 mL of 1×PBS and incubated at 41° C. Aliquots (100 μL) were collected from each sample after 1, 2, and 3 hours of exposure to SIF. These aliquots were serially diluted and phage concentrations were measured by standard plaque assay as described above. The experiment was conducted in triplicate for each phage type. Concentrations of viable phages after exposure to SIF or PBS at a given time point were then compared.

C] Phage Release from Microcapsules in SIF

The extent to which phages were released from microcapsules in the intestinal environment was investigated by incubating encapsulated phages first in SGF and then in SIF. 0.5 g of microencapsulated phages (microcapsules) was added to 4.5 mL SGF in duplicate tube sets and incubated at 41° C. for 1 hour with shaking. SGF was then carefully removed via pipetting and microcapsules were combined with either 4.5 mL of SIF or 4.5 mL 1×PBS and incubated at 41° C. with shaking. Aliquots (100 μL) were collected from each sample at 1, 2, and 3 hours following exposure to SIF. The concentrations of released phages in these aliquots were determined using standard plaque assays with phages' respective host APEC serotypes. The experiment was conducted in triplicate with each phage type.

### Phage Treatment of APEC Challenged Chickens:

### A] Selection of Host Strain and Minimum Inhibitory Concentration (MIC)

As non-pathogenic E. coli are among the bacteria normally present in chickens' gastrointestinal tracts, potential antibiotic resistance selection markers in challenge APEC strains were identified by conducting antibiograms of each APEC strain in the library of APEC strains using a previously described micro-broth dilution method. 90 μL of Brain Heart Infusion broth was aliquoted into each well of 96-well plates. Antibiotics (specifically, kanamycin, gentamycin, neomycin, nalidixic acid, streptomycin, and ampicillin) were added to wells in order to establish an antibiotic concentration gradient spanning across each antibiotic's minimum inhibitory concentration (MIC) as reported by NARMS. Each APEC strain was incubated until it reached log-phase growth; these APEC cultures were then diluted at a ratio of 1:20 in fresh BHI broth and aliquots (10 μL) of diluted cultures were added to each well (with only one APEC strain used per phage). Plates were incubated overnight at 37° C. Wells containing only bacterial cultures and BHI broth or only BHI broth were included as negative and contamination controls, respectively. Following incubation, the lowest concentration of each antibiotic that prevented visible bacterial growth was recorded as the MIC for that antibiotic. MICs were confirmed for each APEC strain by plating each strain on sorbitol-MacConkey agar supplemented with antibiotics at the determined MICs and incubating plates overnight at 37° C. The challenge APEC strain was then selected by incubating samples from chicken ceca on sorbitol-MacConkey agar supplemented with antibiotics at the determined MICs and incubating these samples overnight at 37° C. Following these trials, the APEC strain O2:H4 was chosen as the challenge strain for future animal studies because of its resistance to streptomycin and gentamycin at 64 ug/mL and 32 ug/mL, respectively. At these levels, streptomycin and gentamycin were found to prohibit the growth of all other bacteria in the cecal samples.

### B] Bacteriophage Composition and Encapsulation

The bacteriophage composition was prepared by first combining the six bacteriophages in volumes adjusted to ensure a final concentration of 10⁸ PFU/mL per phage. The concentration of the composition was confirmed through standard plaque assay as described above. The phages was then encapsulated using the protocol described above and stored at 4° C. until further use.

### Efficacy of Phage Treatment In Vivo

Chicks were randomly assigned to one of four separate rooms corresponding to one of four treatments (n=26 birds per treatment): 1) unchallenged birds receiving no phage treatment (negative control); 2) APEC challenged birds receiving no phage treatment (positive control); 3) unchallenged birds receiving a mixture of unencapsulated and encapsulated phages; and 4) APEC challenged birds receiving a mixture of unencapsulated and encapsulated phages. All birds were fed a standard diet without medications and were provided with both food and water ad libitum. On day (d) 4 of the experiment, prior to any phage treatment or APEC challenge, six birds from each treatment group were euthanized by CO₂ and cervical dislocation. Cecal contents and lung tissues were collected and assessed for the presence of naturally occurring antibiotic-resistant E. coli strains, as described below.

On day 5, birds in treatment groups receiving a bacterial challenge were administered APEC serotype O2:H4 by oral gavage (1 mL at 10⁷ CFU/mL). On days 4, 5, 6 and 7, birds in phage treatment groups were orally administered 500 μL (5×10⁷ PFU/mL) of unencapsulated phages using a Prima-Tech Bottle Mount Vaccinator (Neogen) and 500 mg (5×10⁷ PFU/g) of encapsulated phages using a Monojet Curved Tip Syringe. All phage-treated birds were administered both unencapsulated and encapsulated phages; considering both encapsulated and unencapsulated phages, phages were administered at an MOI of 10.

On days 6, 7, and 9, 6 birds from each treatment group were euthanized by CO₂ and cervical dislocation. Cecal contents and lung tissues were collected from each euthanized bird. Cecal content samples (0.5 g) were then added to 4.5 mL of Buffered Peptone Water (BPW) and mixed thoroughly by vortexing. The mixed samples were serially diluted and aliquots (100 μL) of each dilution were spread on SMAC agar plates supplemented with streptomycin (64 μg/mL) and gentamycin (32 μg/mL). Plates were incubated overnight at 37° C., and APEC O2:H4 colonies were counted the following day. The efficacy of phage treatment in reducing bacterial concentrations was assessed by comparing concentrations (CFU/mL) of APEC O2:H4 observed in cecal content samples from phage-treated versus untreated birds.

Lung tissues were homogenized in BPW at a ratio of 1:10 (lung tissue to BPW). Homogenized lung samples were then serially diluted and aliquots (100 μL) of these dilutions were spread on SMAC agar plates supplemented with streptomycin (64 μg/mL) and gentamycin (32 μg/mL). Plates were incubated overnight at 37° C. and APEC O2:H4 colonies were enumerated the following day. The efficacy of phage treatment was assessed by analyzing the differences in the concentrations of APEC O2:H4 observed in lung tissues from phage-treated and untreated birds.

### Analysis

Statistical analysis was performed with GraphPad Prism. One-way ANOVA with repeated measures was used to determine if, in comparison to a PBS control solution, either individual phage types or the poly-phage treatment significantly reduced the concentration of host bacteria in liquid media at any particular time point during assessments of lytic capacity. One-way ANOVA was also used to assess the effects of SGF and SIF on the viability of unencapsulated and encapsulated phages. One-way ANOVA with Tukey's multiple comparison test was used to determine whether the concentrations of phages released into SIF from microcapsules significantly differed over time. The concentrations of APEC O2:H4 detected in cecal content and lung samples collected from phage-treated and untreated birds at given time points were also compared using one-way ANOVA with Tukey's multiple comparison test. Differences were considered statistically different at P<0.05.

### Results:

### A] Phage Isolation and Host Range Characterization

Six bacteriophages that were lytic for one or more of the ten APEC strains were isolated from the waste samples tested. All six phages made circular clearings (i.e., plaques) on the bacterial lawn when spotted on LB agar plates overlayed with their respective APEC host strains (Error! Reference source not found.). Through assessments of the lytic spectrum, or host range, of each of these bacteriophages using standard spot assays that employed all ten APEC strains, AHP was found to be highly lytic (degree of lysis=2.7-3.0) against four different APEC strains (three distinct O-serotypes). Phages AKA, MIA, and AHC were each observed to lyse six different APEC strains (five distinct O-serotypes) with varying degrees of lysis (ranging from 1.0 to 3.0), while MP1 and MP2 were each found to lyse two different APEC strains (one O-serotype) with a high degree of lysis (2.7 in both cases, MP1; 3.0 in both cases, MP2). The six isolated phages together exhibited some degree of lytic capacity against nine of the ten APEC strains tested (Error! Reference source not found.).

TABLE 1
Bacteriophages isolated from waste samples and their APEC hosts

| Host | | SEQ ID |
|---|---|---|
| Serotype | Bacteriophage | number |
| E. coli | Escherichia phage vB_Eco_AHP (AHP) | SEQ ID NO: 1 |
| O6:H31 | Escherichia phage vB_Eco_AHC (AHC) | SEQ ID NO: 2 |
| | Escherichia phage vB_Eco_AKA (AKA) | SEQ ID NO: 3 |
| | Escherichia phage vB_Eco_MIA (MIA) | SEQ ID NO: 4 |
| E. coli O2:H4 | Escherichia phage vB_Eco_MP_1 (MP1) | SEQ ID NO: 5 |
| | Escherichia phage vB_Eco_MP_2 (MP2) | SEQ ID NO: 6 |

TABLE 2
Host range analysis of phages AHP, MP1, MP2, AKA,
MIA, and AHC using ten different APEC serotypes

| E. coli | Bacteriophage | | | | | |
|---|---|---|---|---|---|---|
| serotype | AHP | MP1 | MP2 | AKA | MIA | AHC |
| O1:H23 | 3.0 | — | — | 1.0 | 1.0 | 1.0 |
| O78:H51 | — | — | — | 1.0 | 1.0 | 1.0 |
| O78 | — | — | — | 1.3 | 2.0 | 1.7 |
| O2:H6 | 2.7 | 2.7 | 3.0 | 1.7 | 2.3 | 2.0 |
| O6:H31 | 3.0 | — | — | 3.0 | 2.7 | 2.7 |
| O1:H7 | 3.0 | — | — | — | — | — |
| O5 | — | — | — | 1.7 | 1.7 | 1.7 |
| O2:H4 | — | 2.7 | 3.0 | — | — | — |
| O78:H9 | — | — | — | — | — | — |
| O2 | — | — | — | 2.7 | 3.0 | 3.0 |

— indicates no lysis at all, 0.1-1.0 indicates that the phage is semi-lytic, 1.1-2.0 indicates moderate lysis of bacteria, 2.1-3.0 indicates complete lysis of bacteria and formation of a transparent, circular clearing on the bacterial lawn.

### B] Lytic Capacity of Individual Phages and of a Bacteriophage Composition

The ability of the six isolated bacteriophages to lyse their respective host APEC serotypes in liquid media was investigated by inoculating APEC cultures with the appropriate phage type, incubating the inoculated cultures at 37° C., and measuring absorbance (OD₆₀₀) over time. After 1 hour of incubation, the OD₆₀₀ readings of MP1-treated (0.101), MP2-treated (0.328), and AHP-treated (0.284) APEC cultures were significantly lower (P<0.05) than those of untreated APEC host cultures (untreated MP1 and MP2 host, OD₆₀₀=0.454; untreated AHP host, OD₆₀₀=0.497). The OD₆₀₀ readings of AKA-, MIA-, and AHC-treated cultures did not significantly differ (P>0.05) from those of untreated APEC host cultures after 1 hour of incubation.

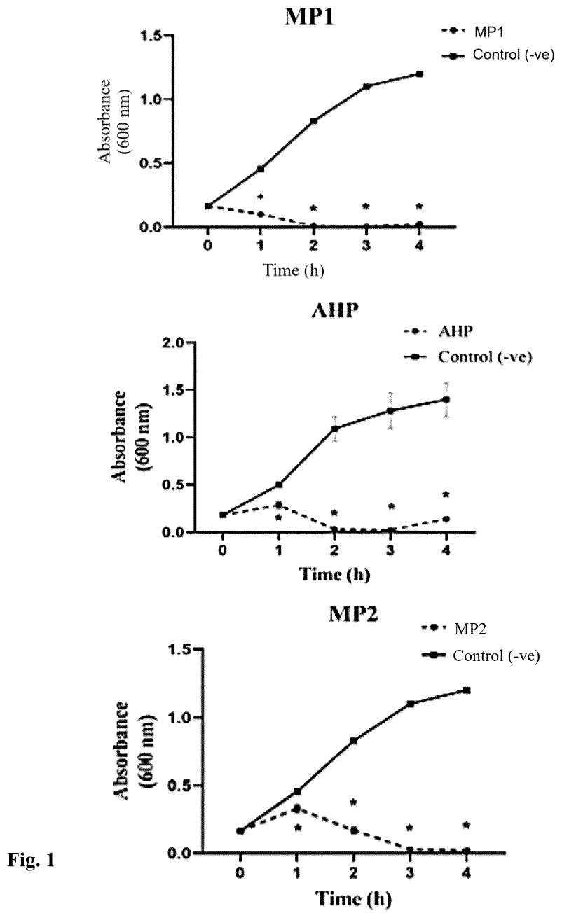

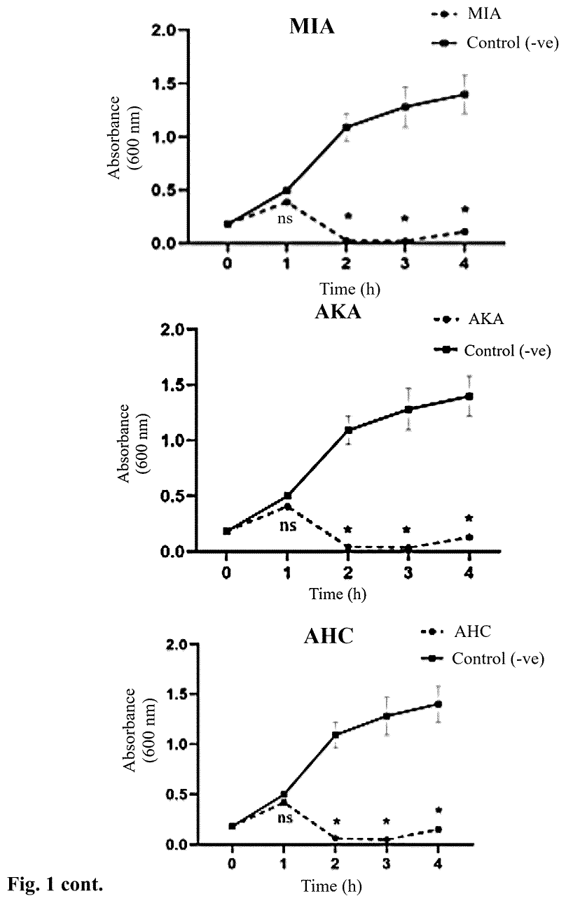

At 2, 3, and 4 hours of incubation, the OD₆₀₀ readings of all phage-treated APEC cultures were significantly lower (P<0.05) than those of untreated APEC host cultures at the same time points. The significantly lower OD₆₀₀ readings observed in all phage-treated APEC cultures at and after 2 hour of incubation indicated that phage treatment resulted in bacterial cell lysis in these cultures (FIG. 1).

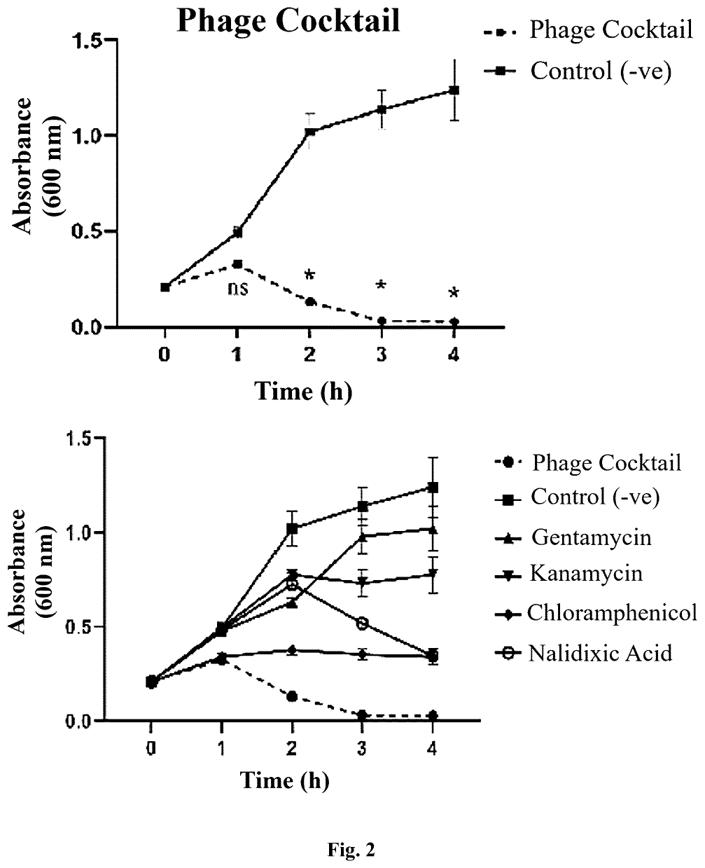

APEC serotype O2:H4 was selected to serve as the host APEC serotype in assessments of the lytic capacity of a phage treatment comprised of all six phages. Antibiotic (specifically, kanamycin, gentamycin, chloramphenicol, and nalidixic acid) treatments were included in these experiments as positive controls. After 2, 3, and 4 hours of incubation, the phage-treated APEC culture (0.131) was significantly lower (P<0.05) than that of the untreated bacterial culture (1.02) at the same time point. These significant differences indicated that phage-induced bacterial lysis occurred in phage-treated APEC cultures. The OD₆₀₀ readings of APEC cultures were treated with chloramphenicol and nalidixic acid, while the OD₆₀₀ readings of cultures treated with gentamycin and kanamycin increased over time (FIG. 2).

### C] Encapsulation Efficiency

The efficiency of encapsulation was determined by comparing the concentration of phages, where phages are encapsulated, to the concentration of phages where phages are not encapsulated (pre-encapsulation phage concentration). Phage AHP had an encapsulation efficiency of 97.9%, phages MP1 and MP2 had encapsulation efficiencies of 98.9% each, and phage AKA had encapsulation efficiency of 98.8%. Phage MIA had an encapsulation efficiency of 95.5%, and phage AHC had an encapsulation efficiency of 94.4%. Overall encapsulation efficiencies of all six phages ranged from 94.4% to 98.9% (see Table 3).

TABLE 3
Encapsulation efficiency of phages MP1, MP2, AKA,
AHP, MIA, and AHC

| | Pre- | Concentration | |
|---|---|---|---|
| | encapsulation | of | Encap- |
| | phage | phages in | sulation |
| | concentration | microcapsules | efficiency |
| Bacteriophages | (PFU/mL) | (PFU/mL) | (%) |
| AHP | 9.6 ± 0.1 | 9.4 ± 0.1 | 97.9 |
| MP1 | 9.3 ± 0.2 | 9.2 ± 0.2 | 98.9 |
| MP2 | 9.4 ± 0.1 | 9.3 ± 0.2 | 98.9 |
| AKA | 8.6 ± 0.2 | 8.5 ± 0.1 | 98.8 |
| MIA | 8.9 ± 0.1 | 8.5 ± 0.1 | 95.5 |
| AHC | 8.9 ± 0.1 | 8.4 ± 0.2 | 94.4 |

### D] Phages Stability in SGF

The stability of both unencapsulated and encapsulated phages was tested in SGF (pH 2.2-2.5) containing protease (specifically, pepsin). In testing with unencapsulated phages, however, concentrations of MP1 and MP2 phages in samples exposed to SGF were observed to be significantly lower (P<0.05) than those in samples exposed to a PBS control solution at all time points (5 minutes post-exposure: concentrations of SGF-exposed MP1 and MP2 was 8.71 log₁₀ PFU/mL and 7.72 log₁₀ PFU/mL, respectively, concentrations of PBS-treated MP1 and MP2 was 9.27 log₁₀ PFU/mL and 9.05 log₁₀ PFU/mL, respectively). Concentrations of MP1 and MP2 phages in samples exposed to SGF for 90 min were 3.9 log₁₀ PFU/mL and 4.5 log₁₀ PFU/mL lower, respectively, than concentrations of the same phages in samples exposed to PBS for 90 minutes. No significant differences (P>0.05) in phage concentrations were observed between SGF-exposed and PBS-exposed AHP samples after 5 and 15 minutes of exposure. Phage concentrations in SGF-exposed AHP samples were found to be significantly lower (P<0.05) than those in PBS-exposed AHP samples after 30, 60, and 90 minutes of exposure. After 90 minutes of exposure, AHP concentrations in SGF-exposed samples were 2.35 log₁₀ PFU/mL lower than those in PBS-exposed samples. AKA, MIA, and AHC phages could not be detected after 5 minutes of exposure to SGF, and phage concentrations in SGF-exposed AKA, MIA, and AHC samples were significantly lower (P<0.05) than those in PBS-exposed AKA, MIA, and AHC samples, respectively, at every time point Concentration of the same phages when exposed to PBS remained unchanged (FIG. 3).

When encapsulated phages were used, in contrast, SGF exposure typically only resulted in significantly lower (P<0.05) phage concentrations in SGF-exposed samples versus PBS-exposed controls after 90 minutes. The concentrations of phages in encapsulated MP1 and MP2 samples exposed to SGF for 60 minutes (MP1: 9.09 log₁₀ PFU/mL; MP2: 9.11 log₁₀ PFU/mL) were not found to be significantly different (P>0.05) than those observed in samples of encapsulated MP1 and MP2 phages exposed to PBS for the same time interval (MP1: 9.23 log₁₀ PFU/mL; MP2: 9.15 log₁₀ PFU/mL). However, after 90 minutes of exposure, phage concentrations in SGF-exposed samples of encapsulated MP1 (8.11 log₁₀ PFU/mL) and of encapsulated MP2 (8.41 log₁₀ PFU/mL) were significantly (P<0.05) lower than those in PBS-exposed samples of encapsulated MP1 (9.19 log₁₀ PFU/mL) and MP2 (9.07 log₁₀ PFU/mL), respectively. Nonetheless, concentrations of both phage types were less than 1.00 log₁₀ PFU/mL lower (MP1: 0.97 log₁₀ PFU/mL lower; MP2: 0.74 log₁₀ PFU/mL lower) in SGF-exposed samples than in PBS-exposed samples after 90 minutes of exposure. By comparison, phage concentrations were 3.9 log₁₀ PFU/mL (MP1) and 4.5 log₁₀ PFU/mL (MP2) lower in SGF-exposed samples than in PBS-exposed controls after 90 minutes of exposure when unencapsulated phages were used. After 60 minutes of exposure, phage concentrations in SGF-exposed samples of encapsulated AHP (9.21 log₁₀ PFU/mL), AKA (8.17 log₁₀ PFU/mL), MIA (8.14 log₁₀ PFU/mL), and AHC (8.13 log₁₀ PFU/mL) were not found to be significantly different (P>0.05) than those observed in samples exposed to PBS (AHP: 9.23 log₁₀ PFU/mL; AKA: 8.27 log₁₀ PFU/mL; MIA: 8.27 log₁₀ PFU/mL; AHC: 8.31 log₁₀ PFU/mL). However, phage concentrations in SGF-exposed samples of encapsulated AHP (8.12 log₁₀ PFU/mL), AKA (7.3 log₁₀ PFU/mL), MIA (7.34 log₁₀ PFU/mL), and AHC (7.15 log₁₀ PFU/mL) were significantly lower (P<0.05) than those in PBS-exposed samples of encapsulated AHP (9.19 log₁₀ PFU/mL), AKA (8.22 log₁₀ PFU/mL), MIA (8.27 log₁₀ PFU/mL), and AHC (8.23 log₁₀ PFU/mL) after 90 minutes of exposure (FIG. 4).

### E] Phages Stability in SIF

The stability of unencapsulated phages in SIF was determined by incubating unencapsulated phages with either SIF or 1×PBS SIF and 1×PBS (control) for 1, 2, and 3 hours. Except in the case of MP1, no significant differences in phage concentrations were observed when unencapsulated phages were incubated with SIF versus without SIF at any time point. Concentrations of MP1 in SIF-exposed samples were significantly lower (P<0.05) than concentrations of MP1 in PBS-exposed samples at all sampling points. (Error! Reference source not found.).

F] Phage Release from Microcapsules in SIF

The release of phages from microcapsules during exposure to SIF following SGF exposure was measured by incubating phage microcapsules in SGF for 1 hour and then incubating them in SIF for 3 hours. The concentration of phages released into the solution was measured after 1, 2 and 3 hours of incubation in SIF. After 1 hour of incubation in SIF, the concentrations of released phages were 9.14 log₁₀ PFU/mL (AHP), 8.94 log₁₀ PFU/mL (MP1), 8.88 log₁₀ PFU/mL (MP2), 7.99 log₁₀ PFU/mL (AKA), 8.05 log₁₀ PFU/mL (MIA) and 7.99 log₁₀ PFU/mL (AHC), suggesting that 97.4% (AHP), 96.5% (MP1), 95.4% (MP2), 94.4% (AKA), 94.7% (MIA) and 95.8% (AHC) of phages were released from their microcapsules after being incubated in SIF for 1 hour following 1 hour of exposure to SGF. The concentration of phages released into the solution were significantly higher (P<0.05) after 2 hours of incubation in SIF than after 1 hour of incubation in SIF in most cases. No significant difference (P>0.05) was observed between the concentration of released phages after 2 hours of incubation in SIF and after 3 hours of incubation in SIF for any phage type. After 3 hours of incubation in SIF, between 97.4% and 99.6% of phages were released from the microcapsules into the SIF solution across all phage types (Error! Reference source not found.).

### G] Efficacy of Phage Treatment In Vivo

The efficacy of phage treatment in vivo was assessed by treating APEC-challenged chickens with a mixture of encapsulated and unencapsulated phages and subsequently enumerating APEC concentrations in ceca and lungs. Importantly, APEC was not detected in either cecal content or lung samples collected from birds that were euthanized prior to the APEC challenge, indicating that the birds used in the study were APEC-free prior to the challenge. There were no significant differences in APEC concentrations in the cecal contents of challenged, phage-treated (5.02 log₁₀ CFU/g) versus challenged, untreated (5.05 log₁₀ CFU/g) chickens were observed at 1 d post-challenge. Conversely, concentrations of APEC in the lungs of challenged, phage-treated chickens (0.69 log₁₀ CFU/g) were significantly (P<0.05) lower than those in the lungs of challenged, untreated chickens (2.39 log₁₀ CFU/g). At 1 day post-challenge, APEC was detected in the lungs of 2 out of 6 chickens in the challenged, phage-treated group and 4 out of 6 chickens in the challenged, untreated group. At this time point, APEC was not detected either in the cecal contents, or lung samples from chickens in the unchallenged, untreated, and the unchallenged, phage-treated groups.

At 2 days post-challenge, concentrations of APEC were significantly lower (P<0.05) in the cecal contents of challenged, phage-treated chickens (4.62 log₁₀ CFU/g) than in the cecal contents challenged, untreated chickens (5.33 log₁₀ CFU/g). Similarly, concentrations of APEC in the lungs of challenged, phage-treated chickens (0.67 log₁₀ CFU/g) were significantly lower than those in the lungs of challenged, untreated chickens (3.15 log₁₀ CFU/g) at 2 days post-challenge. APEC was detected in the lungs of 2 out of 6 chickens in the challenged, phage-treated group and 5 out of 6 chickens in the challenged, untreated group at this time point. APEC was not detected in either the cecal contents, or lungs of chickens in the unchallenged untreated and the unchallenged phage-treated groups at 2 days post-challenge.

At 4 days post-challenge, concentrations of APEC were significantly lower (P<0.05) in the cecal contents of challenged, phage-treated chickens (2.79 log₁₀ CFU/g) than in the cecal contents of challenged, untreated chickens (6.1810 log CFU/g). Concentrations of APEC in the lungs of challenged, phage-treated chickens (0.00 log₁₀ CFU/g) were also significantly lower (P<0.05) than those in the lungs of challenged, un-treated chickens (4.81 log₁₀ CFU/g). In fact, concentrations of APEC were below the detection limits in all lung samples from challenged, phage-treated chickens at 4 days post-challenge. At this time point, APEC was not detected in the cecal contents of unchallenged, phage-treated chickens but was, detected (2.0 log₁₀ CFU/g) in the cecal contents of one chicken in the unchallenged untreated group. APEC was not detected in lung samples from either unchallenged, phage-treated or unchallenged, untreated birds at 4 days post-challenge (Table 4).

TABLE 4
Concentrations of E. coli recovered from cecal contents and lung samples of chickens in all
treatment groups

| | E. coli concentration (log₁₀ CFU/g) | | | | | |
|---|---|---|---|---|---|---|
| | 1 day post-challenge | | 2 days post-challenge | | 3 days post-challenge | |
| Treatment | Cecal | | Cecal | | Cecal | |
| groups | contents | Lungs | contents | Lungs | contents | Lungs |
| 1 | 0.0 ± 0.0 | 0.0 ± 0.0 | 0.0 ± 0.0 | 0.0 ± 0.0 | 0.2 ± 0.7 | 0.0 ± 0.0 |
| 2 | 5.0 ± 0.5 | 2.4 ± 1.8 | 5.3 ± 0.2 | 3.1 ± 1.5 | 6.1 ± 0.3 | 4.8 ± 0.3 |
| 3 | 0.0 ± 0.0 | 0.0 ± 0.0 | 0.0 ± 0.0 | 0.0 ± 0.0 | 0.0 ± 0.0 | 0.0 ± 0.0 |
| 4 | 5.0 ± 0.3 | 0.7 ± 1.0* | 4.6 ± 0.3* | 0.7 ± 1.3* | 2.8 ± 2.1* | 0.0 ± 0.0* |

Treatment groups were as follows: 1) unchallenged birds receiving no phage treatment (−ve control); 2) APEC challenged birds receiving no phage treatment (+ve control); 3) unchallenged birds receiving a mixture of unencapsulated and encapsulated phages; and 4) APEC challenged birds receiving a mixture of unencapsulated and encapsulated phages.
Asterisks indicate a significant difference at P < 0.05. Statistical comparisons are within the column.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It is intended that the scope of the present compositions and methods be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

We claim:

1. A bacteriophage composition for preventing or treating an infection or a disease caused by Avian Pathogenic E. coli (APEC) comprising an effective amount of at least one phage selected from vB_Eco_AHP (AHP), vB_Eco_AHC (AHC), vB_Eco_AKA (AKA), vB_Eco_MIA (MIA), vB_Eco_MP1 (MP1), and vB_Eco_MP2 (MP2), wherein at least one phage is optionally encapsulated.

2. The bacteriophage composition of claim 1, which comprises encapsulated and unencapsulated phages in a ratio of about 1:1.

3. The bacteriophage composition of claim 1, which comprises phages AHP, AHC, AKA, MIA, MP1, and MP2.

4. The bacteriophage composition of claim 1, wherein the encapsulated phages are encapsulated with a natural polymer.

5. The bacteriophage composition of claim 4, wherein the natural polymer is sodium alginate, chitosan, gum, gelatin, collagen, or a combination of two or more thereof.

6. The bacteriophage composition of claim 4, wherein the natural polymer is sodium alginate.

7. A bacteriophage composition for preventing or treating an infection or a disease caused by Avian Pathogenic E. coli (APEC) comprising an effective amount of at least one isolated phage selected from vB_Eco_AHP (AHP) having a genome comprising a nucleotide sequence of SEQ ID NO: 1, vB_Eco_AHC (AHC) having a genome comprising a nucleotide sequence of SEQ ID NO: 2, vB_Eco_AKA (AKA) having a genome comprising a nucleotide sequence of SEQ ID NO: 3, vB_Eco_MIA (MIA) having a genome comprising a nucleotide sequence of SEQ ID NO: 4, vB_Eco_MP1 (MP1) having a genome comprising a nucleotide sequence of SEQ ID NO: 5, and vB_Eco_MP2 (MP2) having a genome comprising a nucleotide sequence of SEQ ID NO: 6, wherein at least one phage is optionally encapsulated.

8. The bacteriophage composition of claim 7, which comprises phages AHP, AHC, AKA, MIA, MP1, and MP2 comprising the nucleotide sequence of SEQ ID Nos: 1 to 6, respectively.

9. The bacteriophage composition of claim 7, wherein the encapsulated phages are encapsulated with a natural polymer selected from sodium alginate, chitosan, gum, gelatin, collagen, and a combination of two or more thereof.

10. The bacteriophage composition of claim 9, wherein the natural polymer is sodium alginate.

11. A method for preventing or treating an infection or a disease caused by APEC comprising administering to a subject a prophylactically or therapeutically effective amount of a bacteriophage composition comprising at least one phage selected from vB_Eco_AHP (AHP), vB_Eco_AHC (AHC), vB_Eco_AKA (AKA), vB_Eco_MIA (MIA), vB_Eco_MP1 (MP1), and vB_Eco_MP2 (MP2), wherein at least one phage is optionally encapsulated.

12. The method of claim 11, which comprises encapsulated and unencapsulated phages in a ratio of about 1:1.

13. The method of claim 11, which comprises phages AHP, AHC, AKA, MIA, MP1, and MP2.

14. The method of claim 11, wherein the encapsulated phages are encapsulated with a natural polymer selected from sodium alginate, chitosan, gum, gelatin, collagen, and a combination of two or more thereof.

15. The method of claim 14, wherein the natural polymer is sodium alginate.

16. The method of claim 11, wherein the subject is poultry.

17. The method of claim 11, wherein the prophylactically or therapeutically effective amount is from about 10 PFU to about 10¹³ PFU of each phage per ml of composition, in volumes between 0.1 mL to 5 mL per bird per treatment.

18. The method of claim 17, wherein the prophylactically or therapeutically effective amount is administered orally, topically, or subcutaneously to the subject.

19. The method of claim 18, wherein the prophylactically or therapeutically effective amount is administered in a suspension, feed, water, a spray, or an injection to the subject.

20. The method of claim 17, wherein the prophylactically or therapeutically effective amount is administered to the subject over a period of time ranging from about 1 to about 60 days.
